(19) United States
(12) Patent Application Publication
(10) Pub. No.: US 2020/0166507 A1
(43) Pub. Date: May 28, 2020

(54) MEASUREMENT METHOD AND MEASUREMENT KIT FOR PROTEINS RELATED TO GENETIC DISEASES

(71) Applicant: Nobelpharma Co., Ltd., Tokyo (JP)

(72) Inventors: Yoshiki YAGI, Tokyo (JP); Yoshinori YUKUTAKE, Tokyo (JP)

(21) Appl. No.: 15/998,549

(22) PCT Filed: Feb. 13, 2017
(86) PCT No.: PCT/JP2017/005198

§ 371 Date: Feb. 11, 2020

(30) Foreign Application Priority Data
Feb. 15, 2016 (JP) 2016-026200

(51) Int. Cl. G01N 33/553; G01N 21/76; G01N 33/531

(52) U.S. Cl. CPC G01N 33/553; G01N 33/531; G01N 21/76

(57) ABSTRACT

Object:

To provide a protein measurement and the like having excellent sensitivity, trueness, and precision.

Resolution means:

A protein measurement method including: an antigen binding step S16 in which a protein related to a genetic disease is made to bind with a capture antibody that recognizes said protein; a detection antibody binding step S17 in which said protein bound to said capture antibody is made to bind with a detection antibody that recognizes a region different from the region recognized by said capture antibody of said protein and that is labeled with a luminescent metal complex; and a measurement step S19 in which said detection antibody bound to said protein and labeled with said luminescent metal complex is detected by measuring the electrochemiluminescence generated by electrochemical stimulation.

### TECHNICAL FIELD

The present invention relates to a measurement method for proteins related to genetic diseases contained in a biological sample, and a measurement kit using the same.

### BACKGROUND ART

A genetic disease is a disease caused by a variation in a gene or chromosome. At present, more than 10000 genetic diseases are known. Known genetic variations include deletion mutations, duplication mutations, missense mutations, and nonsense mutations. Depending on the mutations, disease symptoms are exhibited due to a protein losing its original function because the normal protein is not expressed or because translation of the protein stops midway. Substantially, all diseases known as intractable or rare diseases are classified as genetic diseases, the majority of which are serious diseases that are difficult to cure. The development of therapeutic drugs and therapeutic methods that treat such genetic diseases by promoting restoration of protein expression is being actively pursued. For example, exon skipping therapy using an antisense drug for missense mutations and read-through therapy using a read-through drug for nonsense mutations are being developed. Alternatively, gene therapy in which a normal gene is transferred and expressed, and nucleic acid drugs such as siRNA, decoy nucleic acids, aptamers, and ribozymes are being developed.

Muscular dystrophy is one example of a genetic disease. Muscular dystrophy is a genetic disease of which the main pathology is degeneration or necrosis of skeletal muscles. It presents clinically as a progressive decrease in muscle strength. The cause of Duchenne muscular dystrophy (DMD), which is one type of muscular dystrophy, is defective expression of dystrophin protein (also referred to simply as “dystrophin” hereinafter) caused by a dystrophin gene deletion mutation. In treatment of DMD, development of therapies that promote restoration of dystrophin expression using antisense drugs and read-through drugs is progressing (for example, refer to Non-Patent Documents 1, 2). Development of gene therapy is also progressing. Studies of these therapies are being actively conducted in Japan and other countries. In these studies, the restoration of dystrophin expression in patient tissue is evaluated after compound drug administration or after gene transfer. Assays of dystrophin solubilized from patient tissues are used to ascertain restoration of dystrophin expression. As described in Non-Patent Documents 1 and 2, western blotting is used as the assay.

### CITATION LIST

### Non-Patent Documents

Non-Patent Document 1: Lancet. 2011, 378 (9791), 595-605.

Non-Patent Document 2: The New England Journal of Medicine. 2011, 364, 1513-1522.

Non-Patent Document 3: European Medicines Agency. Guideline on the clinical investigation of medicinal products for the treatment of Duchenne and Becker muscular dystrophy. 2015.

Non-Patent Document 4: Food and Drug Administration. Duchenne Muscular Dystrophy and Related Dystrophinopathies: Developing Drugs for Treatment Guidance for Industry, Draft Guidance. 2015.

### SUMMARY OF INVENTION

### Technical Problem

In therapies using antisense drugs or read-through drugs or gene therapy as described above, there are many cases in which restoration of only a negligible amount of protein compared to healthy people is expected based on the pharmacological action mechanism. However, since expression of the causative protein is fundamentally nonexistent in patients with serious genetic diseases, some extent of symptom recovery is expected by restoring even a few tenths of the amount of protein expression of a healthy person. Alternatively, symptom recovery and suppression of progression is expected through even a slight suppression of abnormal protein accumulation. For this reason, a method that can measure minute amounts of protein is needed in order to ascertain restoration of normal protein expression and to ascertain suppression of abnormal protein accumulation in studies on genetic diseases.

Western blotting is sometimes used as a protein measurement method in studies at present, but this method has the problems that the procedure is complex, irregularity of measurement results is large, and precision is poor. Furthermore, known high-precision measurement methods include detection methods using liquid chromatography/tandem mass spectrometry (LC/MS/MS), chemiluminescence, and the like, but these methods have the problem that they have great irregularity and lack precision because their procedures are complex.

Another known protein measurement method is ELISA. ELISA kits are commercially available, the procedure is relatively low in complexity, and the method has high precision. With ELISA, however, sensitivity drops greatly in the presence of high concentrations of surfactants and reducing agents, and as a result, the amount of surfactants and reducing agents contained in the sample solution needs to be kept to a minimum. For example, in DMD therapy, high concentrations of surfactants and reducing agents are used to solubilize the biological sample because dystrophin is a highly hydrophobic protein located near membranes. ELISA is not suitable for measuring such hydrophobic proteins because its sensitivity drops markedly in the presence of high concentrations of surfactants and reducing agents. Furthermore, ELISA has the problem that the sensitivity of the color development method used as a detection method is low, and it is not suitable for genetic disease studies in which trace amounts of protein need to be measured. For these reasons, the use of ELISA in studies on DMD therapy has not been reported.

With this as a background, the draft guideline of the European Medicines Agency points out that current dystrophin assays are not sufficiently robust (Non-Patent Document 3). Additionally, the draft guidance of the U.S. Food and Drug Administration states that the measurement method needs improvement (Non-Patent Document 4). Thus, a measurement method for proteins related to genetic diseases having high specificity and excellent sensitivity, trueness, and precision and that can withstand examination for pharmaceutical approval is needed.

### Solution to Problem

The present invention was achieved while taking this background art into consideration. An object thereof is to provide a measurement method for proteins related to genetic diseases having excellent sensitivity, trueness, and precision.

As a result of diligent research on the above problem, the present inventors discovered that measurement with excellent sensitivity, trueness, and precision is enabled by utilizing electrochemiluminescence (ECL) in measurement of proteins related to genetic diseases, and completed the present invention.

The present invention provides the specific aspects of (1) to (17) below. (1) A measurement method for proteins, the method including: an antigen binding step of binding a protein related to a genetic disease with a capture antibody recognizing the protein; a detection antibody binding step of binding the protein bound to the capture antibody with a detection antibody recognizing a region of the protein different from a region recognized by the capture antibody and labeled with a luminescent metal complex; and a measurement step of detecting the detection antibody bound to the protein and labeled with the luminescent metal complex by measuring an electrochemiluminescence level generated by electrochemical stimulation.

(2) The measurement method for proteins according to the above (1), wherein the capture antibody is a monoclonal antibody and the detection antibody is a polyclonal antibody.

(3) The measurement method for proteins according to the above (1), wherein the capture antibody is a polyclonal antibody and the detection antibody is a monoclonal antibody.

(4) The measurement method for proteins according to any one of the above (1) to (3), wherein the capture antibody is an antibody recognizing a C-terminal region of the protein and the detection antibody is an antibody recognizing an N-terminal region of the protein.

(5) The measurement method for proteins according to any one of the above (1) to (3), wherein the capture antibody is an antibody recognizing an N-terminal region of the protein and the detection antibody is an antibody recognizing a C-terminal region of the protein.

(6) The measurement method for proteins according to any one of the above (1) to (5), the method further including: a solubilizing step of solubilizing the protein from a biological sample using a solubilizing solution containing a surfactant and a reducing agent; and a diluting step of diluting the solubilized sample solution obtained in the solubilizing step to obtain a dilute sample solution containing the protein; wherein, in the antigen binding step, the protein contained in the dilute sample solution is made to bind with the capture antibody.

(7) The measurement method for proteins according to the above (6), wherein, in the solubilizing step, cells or tissues derived from muscle, brain, blood, or heart, or cells or tissues cultured or derived from stem cells derived from biological cells are used as the biological sample.

(8) The measurement method for proteins according to the above (6) or (7), wherein the surfactant of the solubilizing solution used in the solubilizing step contains an anionic surfactant.

(9) The measurement method for proteins according to any one of the above (6) to (8), wherein, in the diluting step, the solubilized sample solution is diluted using a diluting solution containing a nonionic surfactant.

(10) The measurement method for proteins according to any one of the above (6) to (9), the method further including: a calculation step of calculating a quantity of the protein contained in the biological sample based on a standard curve of concentration and electrochemiluminescence level of a standard protein and an electrochemiluminescence level of the protein contained in the biological sample.

(11) The measurement method for proteins according to the above (10), wherein a full length of the protein or a portion thereof is used as the standard protein.

(12) The measurement method for proteins according to any one of the above (1) to (11), wherein the luminescent metal complex is introduced to the detection antibody by a detection secondary antibody recognizing the detection antibody and labeled with the luminescent metal complex.

(13) The measurement method for proteins according to any one of the above (1) to (12), the method further including: a secondary antibody immobilizing step of immobilizing an immobilizing secondary antibody recognizing the capture antibody; and a capture antibody binding step of immobilizing the capture antibody by binding the immobilized immobilizing secondary antibody with the capture antibody.

(14) The measurement method for proteins according to the above (13), wherein the immobilizing secondary antibody is immobilized to a solid phase, and the solid phase is a plate, a cuvette, a tube, beads, a porous article, or a membrane.

(15) The measurement method for proteins according to any one of the above (1) to (14), wherein the protein is dystrophin protein.

The present invention also provides the specific aspects of (16) and (17) below.

(16) A protein measurement kit for ECL, the kit containing: a capture antibody recognizing a protein related to a genetic disease; and a detection antibody recognizing a region of the protein different from the region recognized by the capture antibody and labeled with a luminescent metal complex.

(17) The protein measurement kit for ECL according to the above (16), the kit further containing: a detection secondary antibody recognizing the detection antibody and labeled with the luminescent metal complex.

### Advantageous Effects of Invention

According to the present invention, provided is a measurement method for proteins related to genetic diseases having excellent sensitivity, trueness, and precision.

### BRIEF DESCRIPTION OF DRAWINGS

FIG. 1 is a flowchart illustrating an example of a measurement method for dystrophin of the present embodiment.

FIG. 2 is a graph showing a dystrophin standard curve of an example.

### DESCRIPTION OF EMBODIMENTS

Embodiments of the present invention will be described in detail below, but these embodiments are given as examples for describing the present invention, and the present invention is not limited thereto and may be optionally modified within a scope that does not deviate from the spirit of the present invention. Note that in the present specification, a numeric range denoted as, for example, “from 1 to 100” includes both the lower limit “1” and the upper limit “100”. The same is true for denotations of other numeric ranges.

The measurement method for proteins of the present embodiment (also referred to simply as “the present measurement method” hereinafter) includes: an antigen binding step of binding a protein with a capture antibody that recognizes the protein; a detection antibody binding step of binding the protein bound to the capture antibody with a detection antibody that recognizes a region of the protein different from a region recognized by the capture antibody and labeled with a luminescent metal complex; and a measurement step of detecting the detection antibody bound to the protein and labeled with the luminescent metal complex by measuring the electrochemiluminescence level generated by electrochemical stimulation. Specifically, the present measurement method detects a protein utilizing electrochemiluminescence (ECL). Preferably, the present measurement method detects a protein contained in a biological sample.

Additionally, the present measurement method preferably further includes a solubilizing step of solubilizing a protein from a biological sample using a solubilizing solution containing a surfactant and a reducing agent, and a diluting step of diluting the solubilized sample solution obtained in the solubilizing step to prepare a dilute sample solution containing the protein. Furthermore, the present measurement method preferably further includes a standard curve creation step of creating a standard curve of concentration and electrochemiluminescence level of a standard protein, and a calculation step of calculating a quantity of the protein contained in the biological sample based on this standard curve and the electrochemiluminescence level of the protein contained in the biological sample. Additionally, the present measurement method may also include a secondary antibody immobilizing step of immobilizing an immobilizing secondary antibody that recognizes the capture antibody, and a capture antibody binding step of binding the immobilizing secondary antibody with the capture antibody.

The present measurement method is advantageously used in measurement of proteins related to genetic diseases because it enables measurement with excellent sensitivity, trueness, and precision. A genetic disease is a disease caused by an abnormality in one or more genes or chromosomes. In a genetic disease, disease symptoms are exhibited due to decreased protein expression or abnormal protein expression caused by an abnormality in a gene or chromosome. The present measurement method is particularly advantageously used for ascertaining normal protein expression when restoring expression through drug or gene therapy for proteins related to genetic diseases.

Examples of the genetic diseases include, but are not limited to, autosomal dominant diseases such as congenital muscular dystrophy, Marfan syndrome, neurofibromatosis, and sickle-cell anemia, autosomal recessive diseases such as metachromatic leukodystrophy, cystic fibrosis, phenylketonuria, homocystinuria, maple syrup urine disease, and galactosemia, and X-linked inherited diseases such as monogenic diseases including Duchenne muscular dystrophy, Becker muscular dystrophy, adrenoleukodystrophy, and hemophilia; polygenic diseases such as cleft lip, cleft palate, and congenital heart disease; chromosomal anomalies such as Down's syndrome, 4p minus syndrome, and Turner's syndrome, mitochondrial inherited diseases such as chronic progressive external ophthalmoplegia and Leber's disease; and somatic cell genetic diseases such as cancer. Among these genetic diseases, the present measurement method is advantageously used in measurement of proteins related to monogenic diseases in which symptoms can be effectively improved by restoring normal protein expression using antisense drugs, read-through drugs, or gene therapy. More preferably, it is used in measurement of dystrophin related to Duchenne muscular dystrophy or dystrophin related to Becker muscular dystrophy, and even more preferably, it is used in measurement of dystrophin related to Duchenne muscular dystrophy.

Furthermore, due to its high sensitivity, the present measurement method is capable of measurement even when a sample solution containing a protein solubilized using a surfactant or reducing agent has been diluted. Thus, the present measurement method can be advantageously used in measuring proteins that are macromolecular or highly hydrophobic or both. Examples of such proteins that are macromolecular or highly hydrophobic or both include dystrophin, laminin, collagen, dysferlin, titin, nebulin, superoxide dismutase, cytochrome oxidase, actin, and the like. Above all, the present measurement method is advantageously used in measurement of dystrophin.

In the embodiments below, cases where dystrophin is measured are cited as examples, but the protein related to genetic a disease that serves as the object of measurement of the present measurement method is not limited thereto. It is possible to implement the present measurement method by varying it as appropriate according to the protein that is the object of measurement.

### Test Materials, Antibodies, and Reagents

The test materials for performing measurements in the present measurement method are classified into biological samples derived from biological tissue that serve as the objects of measurement and standards that serve as the objects of measurement for obtaining a standard curve. First, these test materials will be described. Then, the antibodies and reagents used in the present measurement method will be described.

### Biological Samples

The biological samples used in the present measurement method are not particularly limited as long as they are biological samples derived from cells, tissues, or test subjects that express dystrophin. For example, biological samples collected from animals such as humans, mice, rats, or dogs as test subjects may be used as biological samples. Specific examples of biological samples include cells or tissues derived from muscle, brain, blood, or heart of these test subjects, but are not particularly limited thereto. Alternatively, the above cells derived from muscle, brain, blood, or heart, or cells or tissues cultured or derived from stem cells derived from biological cells, which have been induced to differentiate so as to express dystrophin, may be used. Examples of the stem cells include embryonic stem cells (ES cells), somatic cell-derived ES cells (ntES cells), induced pluripotent stem cells (iPS cells), and the like, but are not particularly limited thereto. The biological sample is submitted to measurement in the state where dystrophin has been solubilized via a solubilizing step to be described later.

### Standard

The standard (standard dystrophin) is a protein used as a standard sample in the standard curve creation step. The full length of dystrophin, which has a molecular weight of 427 kDa, or a portion thereof may be used. Above all, recombinant full-length dystrophin (RFD) expressed by transferring an expression vector into a mammalian cell, wherein cDNA of the gene (Dp427) that encodes full-length dystrophin of molecular weight 427 kDa has been integrated in the expression vector, is preferably used as the standard dystrophin.

Here, the standard dystrophin may contain the full length of dystrophin of molecular weight 427 kDa, or may contain a portion of dystrophin of molecular weight 427 kDa. In this case, the portion of dystrophin of molecular weight 427 kDa preferably contains at least the recognition site of the capture antibody and the recognition site of the detection antibody. Furthermore, it preferably contains an amino acid sequence that duplicates the site of dystrophin of which expression is to be restored in accordance with the dystrophin expression restoration induced by the antisense drug, read-through drug, or gene therapy administered to the test subject.

### Capture Antibody

The capture antibody is an antibody that specifically recognizes dystrophin and that binds to dystrophin through an antigen-antibody reaction with dystrophin.

The recognition site of the capture antibody on dystrophin is not particularly limited but the capture antibody preferably recognizes and binds to a region different from the detection antibody to be described later. More preferably, the capture antibody and the detection antibody each recognize two or more non-interfering epitopes on dystrophin. Even more preferably, one of either the capture antibody or the detection antibody recognizes the N-terminal region of dystrophin, and the other recognizes the C-terminal region of dystrophin. Specifically, it is preferred that a sandwich immunoassay that binds each to two or more epitopes of dystrophin is performed using the capture antibody and the detection antibody. Particularly preferably, the capture antibody recognizes the C-terminal region of dystrophin and the detection antibody recognizes the N-terminal region of dystrophin, or the capture antibody recognizes the N-terminal region of dystrophin and the detection antibody recognizes the C-terminal region of dystrophin.

Due to the capture antibody or the detection antibody recognizing the C-terminal region of dystrophin, it can bind to dystrophin synthesized up to the C-terminal region. As a result, dystrophin of which expression was restored by read-through or exon skipping can be detected according to the capture antibody or detection antibody that recognizes the C-terminal region. Due to the capture antibody or the detection antibody recognizing the N-terminal region of dystrophin, it can bind to full-length dystrophin of molecular weight 427 kDa which includes the N-terminal region within the dystrophin isoform. As a result, full-length dystrophin can be detected without the capture antibody or detection antibody that recognizes the N-terminal region binding to dystrophin isoforms of molecular weight 260 kDa, 140 kDa, 116 kDa or 71 to 75 kDa that do not include the N-terminal region.

Note that the N-terminal region of dystrophin means a region containing, for example, amino acid numbers 410 to 450 of full-length dystrophin or at least a portion of those amino acids. Note that the C-terminal region of dystrophin means a region containing, for example, amino acid numbers 3669 to 3685 of full-length dystrophin or at least a portion of those amino acids.

The capture antibody may be a monoclonal antibody against dystrophin or a polyclonal antibody against dystrophin. The capture antibody is preferably a monoclonal antibody from the perspective of increasing specificity for dystrophin by avoiding cross-reactions with antigens other than dystrophin that have epitopes identical or similar to the epitopes of dystrophin recognized by the capture antibody. Alternatively, the capture antibody is preferably a polyclonal antibody from the perspective of improving sensitivity through binding of a plurality of capture antibodies to one dystrophin by enabling binding to a plurality of epitopes of dystrophin. From the perspective of obtaining both specificity and sensitivity of dystrophin detection, it is more preferred that the capture antibody is a monoclonal antibody and the detection antibody is a polyclonal antibody or the capture antibody is a polyclonal antibody and the detection antibody is a monoclonal antibody.

Polyclonal antibodies of the capture antibody and the detection antibody to be described later may be produced using a known technique. For example, a host animal such as a mouse, rabbit, rat, goat, sheep, or chicken is immunized by administering full-length dystrophin, a dystrophin fragment, or a peptide equivalent to a dystrophin fragment. Antiserum is collected from the immunized host animal, and the antibody is purified.

Furthermore, monoclonal antibodies of the capture antibody and the detection antibody to be described later may be produced using a known technique. For example, a host animal used in production of the above monoclonal antibody is immunized by administering full-length dystrophin, a dystrophin fragment, or a peptide equivalent to a dystrophin fragment. After immunization, the spleen or lymph nodes are collected from the immunized host animal, and cells that produce the desired antibody are fused with myeloma cells to obtain hybridomas. By cloning the hybridomas, a monoclonal antibody can be produced from the obtained clones.

To obtain a polyclonal antibody of the capture antibody or the detection antibody that recognizes the C-terminal region, it is preferable to produce an antibody by using a peptide equivalent to the C-terminal region of dystrophin in immunization. Furthermore, to obtain a monoclonal antibody of the capture antibody or the detection antibody that recognizes the C-terminal region, it is preferable to produce an antibody by using a peptide equivalent to the C-terminal region of dystrophin in immunization, or to screen the hybridomas using a peptide equivalent to the C-terminal region of dystrophin. Similarly, to obtain a polyclonal antibody or a monoclonal antibody of the capture antibody or the detection antibody that recognizes the N-terminal region, it is preferable to produce an antibody by using a peptide equivalent to the N-terminal region of dystrophin in immunization, or to screen the hybridomas using a peptide equivalent to the N-terminal region of dystrophin.

The capture antibody is preferably submitted to measurement as a capture antibody solution obtained by dissolving the capture antibody in a buffer solution. As the buffer solution, for example, Tris-HCl buffer solution, Tris-glycine buffer solution, phosphate buffer solution, or the like may be used without particular limitation. The pH of the buffer solution is preferably from 6.5 to 8.5, and more preferably from 7 to 8. Furthermore, the buffer solution may contain carrier proteins, salts, and chelating agents for stabilizing the proteins as necessary. Examples of the carrier proteins include bovine serum albumin (BSA), equine serum albumin, ovalbumin, keyhole limpet hemocyanin, and the like, but are not particularly limited thereto. Examples of the salts include NaCl, KCl, and the like, but are not particularly limited thereto. Examples of the chelating agents include ethylenediaminetetraacetic acid (EDTA), glycoletherdiaminetetraacetic acid, and the like, but are not particularly limited thereto. Specific examples of the buffer solutions containing salts include phosphate buffered saline (PBS) and Tris-buffered saline (TBS), but are not particularly limited thereto.

The concentration of the capture antibody solution is not particularly limited but is preferably from 0.01 to 10 μg/mL, more preferably from 0.1 to 5 μg/mL, and even more preferably from 0.5 to 2 μg/mL. By setting the concentration of the capture antibody solution within the above range, measurement sensitivity can be improved.

The capture antibody may be immobilized to a solid phase. In a case where the capture antibody is immobilized to a solid phase, direct immobilization may be performed by binding the capture antibody to the solid phase, or immobilization may be performed via an immobilizing secondary antibody by immobilizing an immobilizing secondary antibody to be described later to a solid phase and binding this immobilizing secondary antibody with the capture antibody. From the perspective of reducing the background in measurement of electrochemiluminescence level, it is preferable to immobilize the capture antibody via an immobilizing secondary antibody.

When the capture antibody is immobilized to a solid phase, the capture antibody and the solid phase may be bound by covalent bonds or noncovalent bonds (for example, hydrophobic interaction, ionic bonds, hydrogen bonds, van der Waals forces, dipole-dipole bonds). Alternatively, using a ligand and a substance that specifically binds to that ligand, as in binding of avidin and streptavidin, they can be bound by labeling one as the capture antibody and labeling the other as the solid phase.

When the capture antibody is immobilized via an immobilizing secondary antibody, the capture antibody and the immobilizing secondary antibody are made to bind through an antigen-antibody reaction. In this case, it is preferred that the immobilizing secondary antibody specifically recognizes and binds to a constant region of the capture antibody.

### Detection Antibody

The detection antibody is an antibody that specifically recognizes a region of dystrophin different from the region recognized by the capture antibody and that binds to dystrophin through an antigen-antibody reaction with dystrophin. The recognition site of the detection antibody on dystrophin is not particularly limited but the detection antibody preferably recognizes and binds to a region different from the capture antibody. The relationship between the detection antibody recognition site and the capture antibody recognition site is as described above in regard to the capture antibody.

The detection antibody may be a monoclonal antibody against dystrophin or a polyclonal antibody against dystrophin. Like the capture antibody, the detection antibody is preferably a monoclonal antibody from the perspective of increasing specificity for dystrophin. Alternatively, the detection antibody is preferably a polyclonal antibody from the perspective of improving sensitivity. The relationship between monoclonal antibodies and polyclonal antibodies in the detection antibody and the capture antibody is as described above in regard to the capture antibody.

In the present measurement method, the detection antibody bound to dystrophin is detected by measuring the electrochemiluminescence level produced by ECL. Electrochemiluminescence by ECL may be performed by using a luminescent metal complex typically used in ECL. This luminescent metal complex is constituted of a polyvalent metal as a central atom and a ligand. Examples of the polyvalent metal contained in the luminescent metal complex include ruthenium, osmium, rhenium, cerium, europium, terbium, ytterbium, and the like. Examples of the ligand include aromatic multidentate ligands such as bipyridyl, substituted bipyridyl, 1,10-phenanthroline, and substituted 1,10-phenanthroline, but are not particularly limited thereto. Among these luminescent metal complexes, a ruthenium-containing compound having ruthenium as the central atom (ruthenium complex) is preferred, and a metal complex of ruthenium and an aromatic multidentate ligand, for example, a bis(2,2′-bipyridyl)ruthenium complex or a tris(2,2′-bipyridyl)ruthenium complex, is more preferred, and a tris(2,2′-bipyridyl)ruthenium complex is even more preferred.

Detection of the detection antibody by ECL may be performed by electrochemiluminescence of the detection antibody labeled with the luminescent metal complex. The luminescent metal complex may be introduced to the detection antibody by directly labeling the detection antibody with the luminescent metal complex, or by binding a detection secondary antibody, which recognizes the detection antibody and is labeled with the above luminescent metal complex, to the detection antibody, or the like. From the perspective of improving measurement sensitivity, it is preferred that a detection secondary antibody labeled with a luminescent metal complex is made to bind with the detection antibody, and measurement is performed by electrochemiluminescence of the luminescent metal complex with which the detection antibody is labeled via the detection secondary antibody. Labeling of the detection antibody or detection secondary antibody with the luminescent metal complex is not limited thereto, and may be performed using, for example, a sulfonated derivative on a multidentate ligand of a luminescent metal complex (for example, SULFO-TAG (trade name) label available from Meso Scale Discovery Inc.).

The detection antibody is preferably submitted to measurement as a detection antibody solution obtained by dissolving the detection antibody in a buffer solution. Examples of the buffer solution that may be used include the buffer solutions cited in the above description of the capture antibody solution.

The concentration of the detection antibody solution is not particularly limited but is preferably from 0.01 to 10 μg/mL, more preferably from 0.1 to 5 μg/mL, and even more preferably from 0.5 to 2 μg/mL. By setting the concentration of the detection antibody solution within the above range, measurement sensitivity can be improved.

### Immobilizing Secondary Antibody

The immobilizing secondary antibody is an antibody that specifically recognizes the capture antibody and binds with the capture antibody through an antigen-antibody reaction. Furthermore, the immobilizing secondary antibody can be immobilized. When immobilizing the immobilizing secondary antibody to a solid phase, it is performed in the same manner as the above immobilization of a capture antibody to a solid phase.

The recognition site of the immobilizing secondary antibody is preferably a constant region of the capture antibody. Furthermore, an antibody against the host animal used in production of the capture antibody is preferably used as the immobilizing secondary antibody. The immobilizing secondary antibody may be a monoclonal antibody against the capture antibody or may be a polyclonal antibody against the capture antibody. The immobilizing secondary antibody is preferably a polyclonal antibody from the perspective that higher sensitivity is obtained by promoting binding of the capture antibody with the immobilizing secondary antibody immobilized to the solid phase by enabling binding to a plurality of epitopes of the capture antibody.

The immobilizing secondary antibody is preferably submitted to measurement as an immobilizing secondary antibody solution obtained by dissolving the immobilizing secondary antibody in a buffer solution. Examples of the buffer solution that may be used include the buffer solutions cited in the above description of the capture antibody solution.

The concentration of the immobilizing secondary antibody solution is not particularly limited but is preferably from 0.1 to 100 μg/mL, more preferably from 1 to 50 μg/mL, and even more preferably from 5 to 20 μg/mL. By setting the concentration of the immobilizing secondary antibody solution within the above range, measurement sensitivity can be improved.

### Detection Secondary Antibody

The detection secondary antibody is an antibody that specifically recognizes the detection antibody and binds with it through an antigen-antibody reaction with the detection antibody. The detection secondary antibody is labeled with a luminescent metal complex used in detection by ECL described above in regard to the detection antibody. As a result, the detection secondary antibody produces electrochemiluminescence by ECL.

As the detection secondary antibody, an antibody against the host animal used in producing the detection antibody is preferably used. The detection secondary antibody may be a monoclonal antibody against the detection antibody or may be a polyclonal antibody against the detection antibody. The detection secondary antibody is preferably a polyclonal antibody from the perspective that higher sensitivity is obtained through binding of a plurality of detection secondary antibodies to one detection antibody by enabling binding to a plurality of epitopes of the detection antibody.

The detection secondary antibody is preferably submitted to measurement as a detection secondary antibody solution obtained by dissolving the detection secondary antibody in a buffer solution. Examples of the buffer solution that may be used include the buffer solutions cited in the above description of the capture antibody solution.

The concentration of the detection secondary antibody solution is not particularly limited but is preferably from 0.01 to 10 μg/mL, more preferably from 0.1 to 5 μg/mL, and even more preferably from 0.5 to 2 μg/mL. By setting the concentration of the detection secondary antibody solution within the above range, measurement sensitivity can be improved.

### Solid Phase

In the present measurement method, an antigen-antibody reaction between the immobilized capture antibody and dystrophin can be induced by immobilizing the capture antibody to a solid phase as described above or by applying a sample solution containing dystrophin to this solid phase. In this case, dystrophin contained in the sample solution can be bound to the capture antibody, and components contained in the sample solution that do not bind to the capture antibody can be isolated. Thus, dystrophin contained in a biological sample can be specifically detected. Note that applying a sample solution means putting the capture antibody and dystrophin contained in the sample solution in contact by dispensing, infusing, or dripping the sample solution on the solid phase.

The solid phase is not particularly limited but a known substrate typically used in ECL and having a carrier surface for immobilizing the capture antibody may be used as appropriate. Examples of the solid phase include plates, cuvettes, tubes, beads, porous articles, or membranes formed of a substance such as a resin, polymer, glass, ceramic, or metal, but are not particularly limited thereto. Above all, a multiwell plate made of plastic and having a pair of electrodes for producing electrochemiluminescence in each well is preferred.

### Solubilizing Solution

The solubilizing solution is an aqueous solution containing at least a surfactant and is used to solubilize dystrophin contained in the biological sample. Dystrophin is a hydrophobic macromolecule and has the property of being hardly soluble in water. Furthermore, dystrophin is present on the interior side of a cell membrane and binds with actin of the cytoskeleton. For this reason, in measuring dystrophin, it is preferable to obtain a solubilized solution by solubilizing dystrophin using a solubilizing solution containing a surfactant.

The surfactant used in solubilizing is not particularly limited but a cationic surfactant, anionic surfactant, amphoteric surfactant, nonionic surfactant, or the like is typically used. Above all, an anionic surfactant is preferably used. Specific examples of the anionic surfactant include sodium dodecyl sulfate (SDS), dodecyl lithium sulfate, sodium cholate, and sodium deoxycholate, but are not particularly limited thereto. Above all, SDS is preferred.

The concentration of the surfactant is not particularly limited but is preferably from 0.1 w/v % to 10 w/v %, more preferably from 0.5 w/v % to 5 w/v %, and even more preferably from 1 w/v % to 3 w/v % relative to the total quantity of solubilizing solution. By setting the surfactant concentration within the above range, dystrophin can be solubilized from the biological sample and sufficient dystrophin for measurement can be obtained, and the influence of excess surfactant on measurement can be suppressed.

The solubilizing solution preferably further contains a reducing agent. Examples of the reducing agent contained in the solubilizing solution include 2-mercaptoethanol (2-ME), 1,4-dithiothreitol, and reduced glutathione, but are not particularly limited thereto. Above all, 2-ME is preferred.

The concentration of the reducing agent is not particularly limited but is preferably from 0.05 w/v % to 5 w/v %, more preferably from 0.1 w/v % to 1 w/v %, and even more preferably from 0.2 w/v % to 0.5 w/v % relative to the total quantity of solubilizing solution. By setting the reducing agent concentration within the above range, oxidation of solubilized dystrophin can be prevented and coagulation of dystrophin due to formation of disulfide bonds can be prevented, and the influence of excess reducing agent on measurement can be suppressed.

The solubilizing solution is preferably a buffer solution containing a surfactant and a reducing agent. Examples of the buffer solution that may be used include the buffer solutions cited in the above description of the capture antibody solution.

### Diluting Solution

The diluting solution is an aqueous solution used in diluting the solubilized sample solution. It contains at least a nonionic surfactant. Because the solubilized sample solution contains a surfactant, there is a possibility of affecting the antigen-antibody reaction and reducing measurement sensitivity in a case where the solubilized sample solution is submitted to measurement unaltered. On the other hand, in a case where the solubilized sample solution is simply diluted, dystrophin may coagulate due to the drop in surfactant concentration. For this reason, in measuring dystrophin, it is preferable to obtain a dilute sample solution by diluting the solubilized sample solution using a diluting solution containing a nonionic surfactant. Because nonionic surfactants have mild properties among surfactants, the concentration of surfactants used in solubilizing can be reduced to suppress the influence on measurement, and solubilization of dystrophin can be maintained.

Examples of the nonionic surfactant contained in the diluting solution include Nonidet (trade name) P-40 (NP-40), Triton (trade name) X-100, Tween (trade name) 20, octyl-β-glucoside, and the like, but are not particularly limited thereto. Above all, NP-40 is preferred.

The concentration of the nonionic surfactant is not particularly limited but is preferably from 0.005 w/v % to 1 w/v %, and more preferably from 0.01 w/v % to 0.1 w/v % relative to the total quantity of diluting solution. By setting the concentration of nonionic surfactant within the above range, the surfactant contained in the solubilized sample solution can be diluted to suppress the influence on measurement, and solubilization of dystrophin can be maintained.

The diluting solution is preferably a buffer solution containing a nonionic surfactant. Examples of the buffer solution that may be used include the buffer solutions cited in the above description of the capture antibody solution.

### Measurement Method

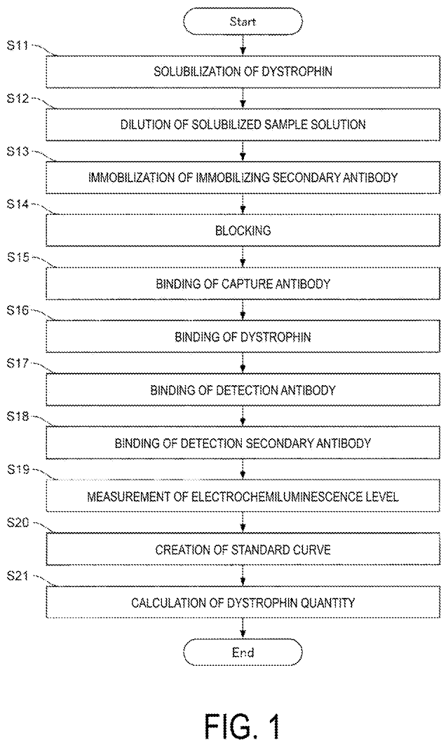

The present measurement method is constituted of an antigen binding step, a detection antibody binding step, and a measurement step according to an operating procedure. The present measurement method preferably also includes a solubilizing step, a diluting step, a secondary antibody immobilizing step, a blocking step, a capture antibody binding step, a detection secondary antibody binding step, a standard curve creation step, and a calculation step. Below, these steps will be described in order in reference to FIG. 1. Note that the present measurement method may be performed using a known electrochemiluminescence level measurement method by ECL.

Note that the present measurement method is described by citing an example in which an immobilizing secondary antibody is immobilized to a solid phase and a capture antibody is made to bind with this immobilizing secondary antibody. Furthermore, it is described by citing an example in which electrochemiluminescence level is measured by inducing electrochemiluminescence of a luminescent metal complex with which the detection secondary antibody is labeled. Additionally, it is described by citing an example in which a monoclonal antibody that recognizes the C-terminal region of dystrophin is used as the capture antibody and a polyclonal antibody that recognizes the N-terminal region of dystrophin is used as a detection antibody. Furthermore, it is described by citing an example in which a welled plate is used as the solid phase.

### Solubilizing Step

First, a biological sample collected from a test subject is solubilized using a solubilizing solution, and a solubilized sample solution in which dystrophin has been solubilized is obtained (step S11: solubilizing step). Here, dystrophin is solubilized together with other proteins contained in the biological sample. The technique for solubilizing the biological sample is not particularly limited but may include, for example, adding the solubilizing solution to the biological sample and then crushing the cells using a homogenizer, glass beads, ultrasonic treatment, or the like while cooling, thereby preparing a cell homogenate of the biological sample. Furthermore, it is preferred that a solubilized sample solution is obtained by centrifuging the homogenate and recovering the supernatant.

### Diluting Step

Next, the solubilized sample solution and a diluting solution are mixed to dilute the solubilized sample solution, and a diluted sample solution is obtained (step S12: diluting step). Here, in the diluted sample solution in the state where dilution has been performed, dilution is preferably performed such that the concentration of surfactant contained in the solubilized sample solution is typically not greater than 1 w/v %, preferably not greater than 0.5 w/v %, and more preferably not greater than 0.2 w/v % relative to the total volume of the diluted sample solution.

### Secondary Antibody Immobilizing Step

Prior to measuring dystrophin by ECL, an immobilizing secondary antibody is immobilized to a solid phase (step S13: secondary antibody immobilizing step). The immobilizing secondary antibody is immobilized by adding an immobilizing secondary antibody solution containing an immobilizing secondary antibody to the wells, and incubating at a certain temperature while shaking. As a result, the immobilizing secondary antibody is immobilized to the solid phase by contacting and binding to the solid phase.

### Blocking Step

Next, blocking is performed on the solid phase to which the immobilizing secondary antibody has been immobilized (step S14: blocking step). Blocking is performed by adding a solution containing a blocking agent such as BSA, skim milk, or casein, which do not bind with the secondary antibody, capture antibody, detection antibody, or detection secondary antibody, to the wells and leaving it to stand. As a result, the solid phase surface is blocked due to the blocking agent contacting and adsorbing to the solid phase. By performing blocking, the antibody and dystrophin can be prevented from nonspecifically adsorbing to the solid phase.

### Capture Antibody Binding Step

The capture antibody is made to bind with the blocked solid phase (step S15: capture antibody binding step). The capture antibody is made to bind by adding a capture antibody solution containing the capture antibody to the wells, and incubating at a certain temperature while shaking. As a result, the capture antibody is immobilized to the solid phase due to the capture antibody and the immobilizing secondary antibody binding through an antigen-antibody reaction.

### Antigen Binding Step

Dystrophin is then made to bind with the capture antibody immobilized to the solid phase (step S16: antigen binding step). Dystrophin is made to bind by adding the diluted sample solution obtained in step S12 to the wells as a measurement sample and incubating at a certain temperature while shaking. As a result, the capture antibody recognizes the N-terminal region of dystrophin and binds through an antigen-antibody reaction.

### Detection Antibody Binding Step

A detection antibody is further made to bind with the dystrophin bound to the capture antibody (step S17: detection antibody binding step). The detection antibody is made to bind by adding a detection antibody solution containing the detection antibody to the wells, and incubating at a certain temperature while shaking. As a result, the detection antibody recognizes the C-terminal region of dystrophin and binds through an antigen-antibody reaction.

### Detection Secondary Antibody Binding Step

Furthermore, a detection secondary antibody is made to bind with the detection antibody bound to dystrophin (step S18: detection secondary antibody binding step). The detection secondary antibody is made to bind by adding a detection secondary antibody solution containing the detection secondary antibody to the wells, and incubating at a certain temperature while shaking. As a result, the detection secondary antibody recognizes the detection antibody and binds through an antigen-antibody reaction. Here, a composite is formed wherein an immobilizing secondary antibody, a capture antibody, dystrophin, a detection antibody, and a detection secondary antibody are bound in that order on a solid phase.

### Measurement Step

After formation of the above composite, the electrochemiluminescence level produced by ECL is measured using an electrochemiluminescence reader (step S19: measurement step). In measurement of the electrochemiluminescence level, first, a solution containing an electron donor substance in the ECL reaction is added to the wells. Additionally, by applying voltage to the solution in the wells via electrodes provided in the wells, electrochemiluminescence is induced from the luminescent metal complex through an oxidation-reduction reaction between the luminescent metal complex with which the detection secondary antibody is labeled and the electron donor substance. The electrochemiluminescence level is measured by detecting this electrochemiluminescence using a photomultiplier provided in the electrochemiluminescence reader. Examples of the electron donor substance include tertiary alkyl amines such as tripropylamine (TPA), but are not particularly limited thereto.

### Standard Curve Creation Step

The electrochemiluminescence level is measured using standard dystrophin in the same manner as the above measurement of electrochemiluminescence level in dystrophin contained in a biological sample using the above biological sample (steps S13 to S19). Here, a standard curve of standard dystrophin concentration and electrochemiluminescence level is created by preparing a plurality of standard sample solutions of different concentrations of standard dystrophin and measuring the electrochemiluminescence levels thereof (step S20: standard curve creation step). Note that in the present specification, the term “standard curve” includes a graph obtained by plotting the relationship between dystrophin concentration and electrochemiluminescence level at each concentration, as well as a regression equation calculated from the relationship between dystrophin concentration and electrochemiluminescence level at each concentration.

### Calculation Step

Additionally, the quantity of dystrophin contained in the biological sample is calculated based on the standard curve obtained in step S20 and the electrochemiluminescence level in dystrophin contained in the biological sample obtained in step S19 (step S21: calculation step).

### Measurement Kit

The protein measurement kit of the present embodiment (also referred to simply as “the present measurement kit” hereinafter) is a kit used for the present measurement method described above, providing the components needed to perform the above measurement method. Specifically, the present measurement kit is a measurement kit for proteins for use with ECL. More specifically, it is a measurement kit for proteins related to genetic diseases.

The present measurement kit includes at least a capture antibody and a detection antibody. The present measurement kit preferably also includes a detection secondary antibody. Additionally, the present measurement kit may include an immobilizing secondary antibody, and may include a solubilizing solution and a diluting solution. Furthermore, the present measurement kit may include an electron donor substance to induce electrochemiluminescence by ECL, and may include RFD used when creating a standard curve. Additionally, the present measurement kit may also include a solid phase, and may include a user's manual on performing the present measurement method.

In the present measurement kit, the capture antibody, detection antibody, detection secondary antibody, immobilizing secondary antibody, electron donor substance, and RFD are preferably aqueous solutions thereof. Furthermore, these aqueous solutions are preferably buffer solutions.

### Action, Effect

The present measurement method measures protein by binding a detection antibody with a protein bound to a capture antibody, and detecting this detection antibody by measuring the electrochemiluminescence level occurring due to electrochemical stimulation. According to the present invention, provided is a measurement method for proteins having excellent sensitivity, trueness, and precision by utilizing ECL. Furthermore, by forming a composite in which the capture antibody, the protein, and the detection antibody are sandwiched in that order, measurement with high specificity for the protein can be performed. As a result, the present measurement method is capable of measuring even trace amounts of protein when ascertaining restoration of expression of proteins related to genetic diseases.

ELISA is known as a measurement method for proteins in the related arts, but the present measurement method can measure with sensitivity from 10 to 100 times greater than that of ELISA. Here, in immunoassay methods such as ELISA, detection sensitivity may drop in the presence of high concentrations of surfactants and reducing agents. For example, with a commercially available kit, the SDS concentration needs to be less than 0.02% and the 2-ME concentration needs to be less than 0.5 mM. Diluting the surfactant and reducing agent as well as the sample solution containing the protein may be considered for avoiding influence by surfactants and reducing agents, but in this case, the signal level drops because the protein concentration decreases through dilution. As a result, there is the problem that measurement is difficult with ELISA. With the present measurement method having high sensitivity, on the other hand, measurement is possible even when the sample solution containing the protein has been diluted, and furthermore, highly sensitive measurement is possible without being affected by surfactants and reducing agents by reducing the concentrations of surfactants and reducing agents by dilution.

The present measurement method has excellent sensitivity compared to western blotting. Furthermore, with western blotting, precision is low because the operations are complex. With the present measurement method, on the other hand, the operations are simple due to performing luminescence by electrochemical stimulation, which also improves precision.

Furthermore, with the present measurement method, due to the fact that the capture antibody or the detection antibody is an antibody that recognizes the C-terminal region of the protein, the capture antibody or detection antibody can recognize and bind to the full-length protein without binding to proteins having defective expression. As a result, when measuring a biological sample derived from the tissue of a DMD patient, it can bind to dystrophin of which expression was restored by antisense drugs, read-through drugs, or gene therapy in cases where exon skipping or read-through occurred. Furthermore, with the present measurement method, due to the fact that the capture antibody or the detection antibody is an antibody that recognizes the N-terminal region of the protein, the capture antibody or the detection antibody can recognize and bind to 427 kDa dystrophin without detecting isoforms of dystrophin. Thus, full-length protein can be detected and measured by combining a capture antibody that recognizes the C-terminal region with a detection antibody that recognizes the N-terminal region, or by combining a detection antibody that recognizes the C-terminal region with a capture antibody that recognizes the N-terminal region. For this reason, according to the present invention, it is possible to ascertain protein expression restoration in test subject tissue after antisense drug or read-through drug administration or after gene therapy.

Furthermore, with the present measurement method, a standard curve is created using the full length of dystrophin of molecular weight 427 kDa or a portion thereof as standard dystrophin, and the quantity of dystrophin is calculated based on this standard curve. Because a commercially available standard obtained by purifying full-length dystrophin does not exist, assay methods in the related art only relatively measure the dystrophin quantity relative to the total protein mass solubilized from biological tissue, and there is the problem that absolute quantitative trueness is not sought. In contrast, according to the present measurement method, trueness can be increased by measuring the absolute amount of dystrophin present.

### EXAMPLES

The present invention will be described in further detail based on various test results, but note that the materials, reagents, used quantities, percentages, treatment details, treatment procedure, and the like described in the following examples may be varied as appropriate as long as they do not deviate from the spirit of the present invention. Thus, the present invention is not limited to the examples below.

Antibodies, Reagents, Devices, and Instruments

### Antibodies

The antibodies used in the examples and reference example are shown in Table 1.

TABLE 1

| | | | Clone/ |
|---|---|---|---|
| Type | Reagent name | Manufacturer | standard |
| Capture | Novocastra Lyophilized | Leica | DY8/6C5 |
| antibody | Mouse Monoclonal | Biosystems | |
| | Antibody Dystrophin | Newcastle | |
| | NCL-DYS2 | Ltd. | |
| Detection | Anti-Dystrophin | Abcam plc. | Unlabeled |
| antibody | antibody ab131315 | | |
| Immobi- | AffiniPure Goat | Jackson | Unlabeled |
| lizing | Anti-Mouse IgG, | Immuno | |
| secondary | Fcγ Subclass 1 | Research | |
| antibody | Specific | Labs | |
| Detection | MSD SULFO-TAG | Meso Scale | SULFO- |
| secondary | labeled Anti-Rabbit | Discovery | TAG |
| antibody | Antibody (Goat) | | label |

The Novocastra (trade name) lyophilized mouse monoclonal antibody dystrophin NCL-DYS2 (referred to as “mAb3” hereinafter) used as the capture antibody is a mouse IgG monoclonal antibody that recognizes the C-terminal region of dystrophin and produces as an immunogen a C-terminal peptide consisting of amino acid numbers 3669 to 3685 of full-length dystrophin.

The anti-dystrophin antibody ab131315 (referred to as “pAb1” hereinafter) used as the detection antibody is a rabbit IgG polyclonal antibody that recognizes the N-terminal region of dystrophin and produces as an immunogen a C-terminal peptide consisting of amino acid numbers 410 to 450 of full-length dystrophin.

The AffiniPure goat anti-mouse IgG, Fcγ subclass 1 specific (referred to as “2nd Ab1” hereinafter) used as the immobilizing secondary antibody is a goat anti-mouse IgG subclass specific polyclonal antibody.

The MSD SULFO-TAG labeled anti-rabbit antibody (goat) (referred to as “SULFO-TAG Ab” hereinafter) used as the detection secondary antibody is a goat anti-rabbit IgG subclass specific polyclonal antibody, which has been labeled with a sulfonated derivative of a tris(2,2′-bipyridyl)ruthenium complex.

### Reagents, Devices, and Instruments

The reagents used in the examples and reference example are shown in Table 2. The devices and instruments used in the examples and reference example are shown in Table 3.

TABLE 2

| Reagent name | Manufacturer | Standard |
|---|---|---|
| 10× D-PBS(-) | Wako Pure Chemical | For cell |
| | Industries, Ltd. | culture |
| 1 M Tris-HCl (pH 6.8) | BioVision, Inc. | |
| SDS Solution, 20% | Serva Electrophoresis | |
| | GmbH | |
| 2-mercaptoethanol | Wako Pure Chemical | Grade 1 |
| | Industries, Ltd. | |
| 20× TBS-T with | Abcam plc. | — |
| Tween 20 | | |
| 10% BSA Diluent/ | Kirkegaard & Perry | — |
| Blocking Solution | Laboratories, Inc. | |
| 1 mol/L Tris-HCl | Nacalai Tesque, Inc. | Biotechnology |
| Buffer Solution | | grade |
| (pH 8.0) | | |
| 0.5 mol/l-EDTA | Nacalai Tesque, Inc. | Biotechnology |
| Solution (pH 8.0) | | grade |
| NP-40 (10% in H₂O) | BioVision Inc. | — |
| MSD Read Buffer T | Meso Scale | — |
| (4×) with surfactant | Discovery | |
| MULTI-ARRAY | Meso Scale | |
| 96-well Bare plate | Discovery | |
| (High Bind) plate for | | |
| ECL immunoassay | | |

TABLE 3

| Device/instrument name | Model | Manufacturer |
|---|---|---|
| Constant temperature | M•BR-022UP | Taitec |
| incubator shaker | | Corporation |
| Programmable low- | IN804 | Yamato |
| temperature constant | | Scientific |
| temperature incubator | | Co., Ltd. |
| Electrochemiluminescence | Sector Imager 6000 | Meso Scale |
| reader | | Discovery |

### Reagent Preparation Examples

### PBS Preparation Example

10 mL of 10×D-PBS(−) and 90 mL of water were mixed. As a result, PBS (1×) was obtained.

### Sodium Chloride Aqueous Solution Preparation Example

5.8 g of sodium chloride was added to a small quantity of water and dissolved, and water was added to make a total volume of 100 mL. As a result, a 1 mol/L sodium chloride aqueous solution was obtained.

### Solubilizing Solution Preparation Example

Water was added to 0.22 mL of 1 mol/L Tris-HCl (pH 6.8), 5 mL of 20% SDS, 2 mL of glycerol, and 2-mercaptoethanol to make a total volume of 50 mL. As a result, a solubilizing solution (4.4 mmol/L Tris, 2% SDS, 4% glycerol, 0.25% 2-ME) was obtained.

### Diluting Solution Preparation Example

2 mL of 1 mol/L Tris-HCl buffer solution (pH 8.0), 15 mL of 1 mol/L sodium chloride aqueous solution, 0.5 mL of NP-40 (10% in H₂O), 10 mL of 10% BSA, and 0.1 mL of 0.5 mol/L EDTA solution (pH 8.0) were mixed. 0.1 mol/L hydrochloric acid was added to adjust the pH to 7.5, and water was then added to make a total volume of 100 mL. As a result, a diluting solution (20 mmol/L Tris, 150 mmol/L sodium chloride, 0.05% NP-40, 1% BSA, 0.5 mmol/L EDTA, pH 7.5) was obtained.

Lavage Fluid Preparation Example 50 mL of 20×TBS-T with Tween 20 (referred to as “20×TBST” hereinafter) and 950 mL of water were mixed. As a result, a lavage fluid (1×TBST) was obtained.

### TBST-BSA Aqueous Solution Preparation Example

50 mL of 10% BSA diluent/blocking solution (referred to as “10% BSA” hereinafter), 5 mL of 20×TBST, and 45 mL of water were mixed. As a result, a TBST-BSA aqueous solution (referred to as “TBST-BSA” hereinafter) was obtained.

### Read Buffer Preparation Example

5 mL of MSD read buffer T (4×) with surfactant and 15 mL of water were mixed. As a result, a read buffer (1×) was obtained.

### Immobilizing Secondary Antibody Solution

21 μL of 2nd Ab1 (1.2 mg/mL) and 2499 μL of PBS were mixed. As a result, an immobilizing secondary antibody solution (10 μg/mL) was obtained.

### Capture Antibody Solution

75 μL of mAb3 (36 μg/mL) and 2625 μL of TBST-BSA were mixed. As a result, a capture antibody solution (1 μg/mL) was obtained.

### Detection Antibody Solution Preparation Example

6 μL of pAb1 (500 μg/mL) and 2994 μL of TBST-BSA were mixed. As a result, a detection antibody solution (1 μg/mL) was obtained.

### Detection Secondary Antibody Solution Preparation Example

6 μL of SULFO-TAG Ab (500 μg/mL) and 2994 μL of TBST-BSA were mixed. As a result, a detection secondary antibody solution (1 μg/mL) was obtained.

### Test Material Preparation Example

### Standard Dystrophin Preparation Example (RFD)

A recombinant full-length dystrophin (RFD) solution (10 fmol/μL) supplied from the Japan National Institute of Advanced Industrial Science and Technology was serially diluted with the diluting solution so as to result in 15 levels of RFD concentration from 0.00001 to 0.04 fmol/μL, and 15 standard sample solutions Al to A15 were obtained as shown in Table 4.

### Biological Sample Preparation Example (Human Muscle Tissue Homogenate)

100 μμL of the solubilizing solution was added to muscle tissue derived from healthy humans, and the cells were crushed using zirconia beads and a bead-style cell crusher to prepare a human muscle tissue homogenate. By centrifuging (4° C., 100000×g, 10 min) the homogenate and recovering the supernatant, a solubilized sample solution in which proteins including dystrophin were solubilized was obtained. The protein concentration of this solubilized sample solution was measured using a protein assay kit (trade name 2-D Quant Kit, GE Healthcare Japan Co., Ltd.). The protein concentration of the solubilized sample solution was 630 μg/mL. Then, this solubilized sample solution was serially diluted with the diluting solution so as to result in five levels of protein concentration from 0.4 to 28.8 μg/mL, and five dilute sample solutions (biological sample solutions al to a5) were obtained as shown in Table 5.

### Standard Curve Test Sample Preparation Example

An RFD solution (10 fmol/μL) was serially diluted with the diluting solution so as to result in seven levels of RFD concentration from 0.00150 to 0.0100 fmol/μL, and seven standard sample solutions B1 to B7 were obtained as shown in Table 6.

### Repeatability Validation Sample Preparation Example

An RFD solution (10 fmol/μL) was serially diluted with the diluting solution so as to result in seven levels of RFD concentration from 0.00150 to 0.0100 fmol/μL, and seven repeatability validation sample solutions C1 to C7 were obtained as shown in Table 7.

### Specificity Validation Sample Preparation Example 1

A cerebellum lysate having a protein concentration of 5 mg/mL was diluted with the diluting solution, and a biological sample solution b having a protein concentration of 0.006 fmol/μL was obtained.

### Specificity Validation Sample Preparation Example 2

A DMD patient muscle tissue homogenate having a protein concentration of 19.5 μg/mL was diluted with the diluting solution, and a biological sample solution c having a protein concentration of 0.006 fmol/μL was obtained.

### EXAMPLE 1

### Standard Curve Range Validation and Dystrophin Measurement Measurement of Electrochemiluminescence Level

The electrochemiluminescence level was measured by the following procedure. Note that for the standard sample solutions A1 to A15 and the biological sample solutions a1 to a5, measurement was performed at three points, and the average of the three electrochemiluminescence levels (ECL signals) was taken as the measured value of electrochemiluminescence level of each sample.

(1) 25 μL of immobilizing secondary antibody solution was added to each well of a MULTI-ARRAY 96-well Bare plate (High Bind) plate for ECL immunoassay (referred to simply as “plate” hereinafter) used as a solid phase, and this was shaken for 30 min or more using a constant temperature shake culture device (25° C.). As a result, the immobilizing secondary antibody 2nd Ab1 was immobilized to the solid phase.

(2) The solution in the plate was removed, and a lavage operation was performed, consisting of adding 300 μL of lavage fluid to each well of the plate and removing it, and then the moisture was fully removed, thereby washing out the excess immobilizing secondary antibody solution 2nd Ab1.

(3) 150 μL of TBST-BSA was added to each well of the plate, and it was shaken using a constant temperature shake culture device (25° C.).

(4) It was left to stand for 8 h or more in a programmable low-temperature constant temperature device (set temperature 4° C., storage chamber controlled temperature range: 2.0 to 8.0° C.). As a result, the plate was blocked with BSA.

(5) The solution was removed from the plate, and the moisture was fully removed.

(6) 25 μL of the capture antibody solution was added to each well of the plate, and it was shaken for 30 min or more using a constant temperature shake culture device (25° C.). As a result, the capture antibody solution mAb3 was immobilized to the solid phase by binding to the immobilizing secondary antibody solution 2nd Ab1.

(7) The solution in the plate was removed, and a lavage operation was performed, consisting of adding 300 μL of lavage fluid to each well of the plate and removing it, and then the moisture was fully removed, thereby washing out the excess capture antibody solution mAb3.

(8) 25 μL of standard sample solutions A1 to A15 and biological sample solutions a1 and a5 was added as measurement samples to each well of the plate, and this was shaken using a constant temperature shake culture device (25° C.). As a result, the dystrophin contained in the measurement samples was bound to the capture antibody solution mAb3.

(9) The solution in the plate was removed, and a lavage operation was performed, consisting of adding 300 μL of lavage fluid to each well of the plate and removing it, and then the moisture was fully removed, thereby washing out the excess dystrophin and components contained in the measurement samples that do not bind to the capture antibody solution mAb3.

(10) 25 μL of the detection antibody solution was added to each well of the plate, and it was shaken for 30 min or more using a constant temperature shake culture device (25° C.). As a result, the detection antibody pAb2 was bound to dystrophin.

(11) The solution in the plate was removed, and a lavage operation was performed, consisting of adding 300 μL of lavage fluid to each well of the plate and removing it, and then the moisture was fully removed, thereby washing out the excess detection antibody solution pAb1.

(12) 25 μL of the detection secondary antibody solution was added to each well of the plate, and it was shielded from light and shaken for 30 min or more using a constant temperature shake culture device (25° C.). As a result, the detection secondary antibody SULFO-TAG Ab was bound to the detection antibody pAb1.

(13) The solution in the plate was removed, and a lavage operation was performed, consisting of adding 300 μL of lavage fluid to each well of the plate and removing it, and then the moisture was fully removed, thereby washing out the excess detection secondary antibody solution SULFO-TAG Ab.

(14) 150 μL of read buffer was added to each well of the plate, electrochemiluminescence was induced by an electrochemiluminescence reader, and the electrochemiluminescence level (ECL signal) was measured. The measurement results of standard sample solutions A1 to A15 are shown in Table 4.

TABLE 4

| | | | Standard curve | | Standard curve | |
|---|---|---|---|---|---|---|
| | | | (A1 to A15) | | (A1 to A9) | |
| | | | Back- | | Back- | |
| | RFD | Average | calculated | | calculated | |
| | concentration | ECL | value | Trueness | value | Trueness |
| Sample name | (fmol/μL) | signal | (fmol/μL) | (%) | (fmol/μL) | (%) |
| Standard sample | 0.04 | 59965 | 0.0407 | 1.7 | 0.0406 | 1.4 |
| solution A1 | | | | | | |
| Standard sample | 0.02 | 32299 | 0.0193 | −3.7 | 0.0193 | −3.4 |
| solution A2 | | | | | | |
| Standard sample | 0.01 | 18263 | 0.0102 | 1.7 | 0.0102 | 2.1 |
| solution A3 | | | | | | |
| Standard sample | 0.004 | 8120 | 0.00411 | 2.9 | 0.00411 | 2.8 |
| solution A4 | | | | | | |
| Standard sample | 0.002 | 4398 | 0.00195 | −2.4 | 0.00194 | −2.8 |
| solution A5 | | | | | | |
| Standard sample | 0.001 | 2765 | 0.000998 | −0.2 | 0.000993 | −0.7 |
| solution A6 | | | | | | |
| Standard sample | 0.0004 | 1766 | 0.000404 | 1.1 | 0.000403 | 0.7 |
| solution A7 | | | | | | |
| Standard sample | 0.0002 | 1442 | 0.000206 | 3.0 | 0.000207 | 3.6 |
| solution A8 | | | | | | |
| Standard sample | 0.0001 | 1261 | 0.0000927 | −7.3 | 0.0000961 | −3.9 |
| solution A9 | | | | | | |
| Standard sample | 0.00004 | 1161 | 0.0000283 | −29.3 | — | — |
| solution A10 | | | | | | |
| Standard sample | 0.00002 | 1139 | 0.0000132 | −34.0 | — | — |
| solution A11 | | | | | | |
| Standard sample | 0.00001 | 1129 | 0.0000065 | −35.1 | — | — |
| solution A12 | | | | | | |
| Standard sample | 0.000004 | 1120 | * | * | — | — |
| solution A13 | | | | | | |
| Standard sample | 0.000002 | 1125 | 0.0000032 | 57.9 | — | — |
| solution A14 | | | | | | |
| Standard sample | 0.000001 | 1162 | 0.0000287 | 2771.1 | — | — |
| solution A15 | | | | | | |

* Not calculated
— Excluded from standard curve

### Standard Curve Creation and Evaluation

A standard curve (A1 to A15) was created by calculating a regression equation using the 4-parameter logistic model (weighting: 1/Y2) using the measured values of the ECL signals obtained by measuring the standard sample solutions A1 to A15 as Y and the RFD concentrations (fmol/μL) as X. The standard curve was created using a computer system (trade name: SoftMax Pro (Ver. 5.4), Molecular Devices Inc.). Additionally, the back-calculated values of each standard sample solution and the trueness of the back-calculated values were calculated based on this standard curve (A1 to A15), and the standard curve was evaluated. The evaluation results are shown in Table 4 (standard curve (A1 to A15)). Trueness (%) was calculated by the following formula: (back-calculated value/added concentration)×100. Note that in Table 4, it is surmised that the reason that the back-calculated value of the standard sample solution A13 was not calculated was because the back-calculated value was negative.

As shown in Table 4, the ECL signals of the standard sample solutions A10 to A15 were substantially equal ECL signals. From these results it is understood that detection of RFD by the present measurement method is possible in at least the range of 0.000004 fmol/μL (0.001 fmol) to 0.04 fmol/μL (1 fmol). Furthermore, if A9, of which the trueness of the back-calculated value relative to the standard curve (A1 to A15) is within ±25%, is considered to be the lower limit of measurement, it can be judged that the measurement range is from 0.0001 fmol/μL (0.0025 fmol) to 0.04 fmol/μL (1 fmol), and the lower limit of measurement is 0.0001 fmol/μL (0.0025 fmol).

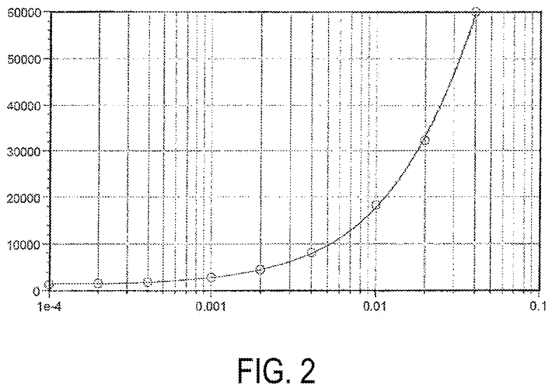

A standard curve (A1 to A9) was created by calculating a regression equation using the 4-parameter logistic model (weighting: 1/Y2) using the measured values of the ECL signals obtained by measuring the standard sample solutions Al to A9 as Y and the RFD concentrations (fmol/μL) as X. This curve is shown in FIG. 2. Note that FIG. 2 shows a standard curve in which dystrophin concentration (fmol/μL) is on the horizontal axis and ECL signal is on the vertical axis. Additionally, the back-calculated values of each standard sample solution and the trueness of the back-calculated values were calculated based on this standard curve (A1 to A9), and the standard curve was evaluated. The evaluation results are shown in Table 4 (standard curve (A1 to A9)). From these results it is understood that the present measurement method has excellent sensitivity and trueness.

### Calculation of Assay Values

The ECL signals obtained by measuring the biological sample solutions a1 to a5 were substituted into the regression equation calculated from the standard sample solutions A1 to A9, and the dystrophin assay values were calculated. The results are shown in Table 5.

TABLE 5

| | Human muscle tissue | | | |
|---|---|---|---|---|
| | homogenate protein | | Dystrophin assay value | |
| | Con- | | Con- | |
| | centration | Quantity | centration | Quantity |
| Sample name | (μg/mL) | (ng) | (fmol/μL) | (fmol) |
| Biological | 0.4 | 10 | 0.000168 | 0.00419 |
| sample | | | | |
| solution a1 | | | | |
| Biological | 1.2 | 30 | 0.000538 | 0.0134 |
| sample | | | | |
| solution a2 | | | | |
| Biological | 4.8 | 120 | 0.00235 | 0.0588 |
| sample | | | | |
| solution a3 | | | | |
| Biological | 14.4 | 360 | 0.00596 | 0.149 |
| sample | | | | |
| solution a4 | | | | |
| Biological | 28.8 | 720 | 0.00529 | 0.132 |
| sample | | | | |
| solution a5 | | | | |

As shown in Table 5, a concentration-dependent increase in dystrophin concentration from a human muscle tissue homogenate protein concentration of 0.4 μg/mL (10 ng) to 14.4 μg/mL (360 ng) was ascertained by the present measurement method. At 28.8 μg/mL (720 ng), it was the same value as the dystrophin concentration at 14.4 μg/mL (360 ng), and it was ascertained that the ECL signal was saturated. However, it was considered that prozone was not seen. From these results it is understood that this is a dystrophin measurement method in which measurement is possible in at least a range of human muscle tissue homogenate dystrophin concentration from 0.4 μg/mL (10 ng) to 14.4 μg/mL (360 ng), the lower limit of the detection range is low, and sensitivity is excellent. Note that 1000 μg is the assay limit in dystrophin measurement by the existing western blotting method, and 100 times that sensitivity is obtained by the present measurement method.

### EXAMPLE 2

### Evaluation of Standard Curve

### Measurement of Electrochemiluminescence Level: First Time

Electrochemiluminescence level was measured in the same manner as Example 1 except that the standard sample solutions B1 to B7 were used as measurement samples instead of the standard sample solutions A1 to A15 and biological sample solutions a1 to a5, and that one point was measured. The electrochemiluminescence levels of one point of the standard sample solutions B1 to B7 were taken as the measured values of each sample. These measured values were used as the data of the standard curve (first time).

### Measurement of Electrochemiluminescence Level: Second, Third Times

In the same environment (staff member, equipment) as the first time of electrochemiluminescence level measurement of Example 2, the electrochemiluminescence levels of the same samples (the standard sample solutions B1 to B7) were measured twice on different days, and the respective measured values were obtained. These measured values were used as the data of the standard curve (second, third times).

### Standard Curve Creation and Evaluation

The standard curves were created for the first to third times of electrochemiluminescence level measurement by calculating regression equations using the 4-parameter logistic model (weighting: 1/Y2) using the measured values of the ECL signals obtained by measuring the standard sample solutions B1 to B7 as Y and the RFD concentrations (fmol/μL) as X. Furthermore, the correlation coefficient of these standard curves was calculated. Additionally, the back-calculated values of each standard sample solution and the trueness of the back-calculated values were calculated based on these standard curves, and the standard curves were evaluated. The results are shown in Table 6 (standard curves (first to third times)).

TABLE 6

| | | Standard curve | | Standard curve | | Standard curve | |
|---|---|---|---|---|---|---|---|
| | | (first time) | | (second time) | | (third time) | |
| | | Correlation coefficient | | | | | |
| | | 0.9997 | | 0.9997 | | 0.9989 | |
| | | Back- | | Back- | | Back- | |
| | RFD | calculated | | calculated | | calculated | |
| Sample | concentration | value | Trueness | value | Trueness | value | Trueness |
| name | (fmol/μL) | (fmol/μL) | (%) | (fmol/μL) | (%) | (fmol/μL) | (%) |
| Standard | 0.000150 | 0.000131 | 87.6 | 0.000178 | 118.5 | 0.000139 | 92.9 |
| sample | | | | | | | |
| solution B1 | | | | | | | |
| Standard | 0.000200 | 0.000217 | 108.5 | 0.000183 | 91.7 | 0.000186 | 92.8 |
| sample | | | | | | | |
| solution B2 | | | | | | | |
| Standard | 0.000400 | 0.000417 | 104.2 | 0.000378 | 94.4 | 0.000458 | 114.4 |
| sample | | | | | | | |
| solution B3 | | | | | | | |
| Standard | 0.00100 | 0.000965 | 96.5 | 0.00101 | 100.7 | 0.000976 | 97.6 |
| sample | | | | | | | |
| solution B4 | | | | | | | |
| Standard | 0.00200 | 0.00204 | 101.8 | 0.00204 | 101.8 | 0.00194 | 96.8 |
| sample | | | | | | | |
| solution B5 | | | | | | | |
| Standard | 0.00400 | 0.00399 | 99.7 | 0.00395 | 98.9 | 0.00413 | 103.3 |
| sample | | | | | | | |
| solution B6 | | | | | | | |
| Standard | 0.0100 | 0.0100 | 100.0 | 0.0100 | 100.2 | 0.00993 | 99.3 |
| sample | | | | | | | |
| solution B7 | | | | | | | |

As shown in Table 6, in measurement of electrochemiluminescence level for a total of three times, the correlation coefficient was 0.9989 or greater, and the trueness of the back-calculated values was from 87.6 to 118.5% for B1 and B7 and from 91.7 to 114.4% for the other standard sample solutions. According to these results, it was ascertained that good standard curves were obtained by calculating the regression equations using the 4-parameter logistic model (weighting: 1/Y2) in the range from 0.000150 fmol/μL to 0.01 fmol/μL, which is the measurement range examined in Example 1.

### EXAMPLE 3

### Repeatability Evaluation

### Measurement of Electrochemiluminescence Level: First Time

Electrochemiluminescence level was measured in the same manner as Example 1 except that the standard sample solutions B1 to B7 and the repeatability validation sample solutions C1 to C7 were used as measurement samples instead of the standard sample solutions A1 to A15 and the biological sample solutions a1 to a5, and that five points were measured. The average electrochemiluminescence level of the five points was taken as the measured value of electrochemiluminescence level of each sample. These measured values were used as data of intraday repeatability and data of the first time of interday repeatability.

### Measurement of Electrochemiluminescence Level: Second, Third Times

In the same environment (staff member, equipment) as the first time of electrochemiluminescence level measurement of Example 3, the electrochemiluminescence levels of the same samples (the standard sample solutions B1 to B7 and the repeatability validation sample solutions C1 to C7) were measured twice on different days, and the respective measured values were obtained. These measured values were used as data of the second time of interday repeatability and data of the third time of interday repeatability.

### Standard Curve Creation and Assay Value Calculation

The standard curves were created for the first to third measurements of electrochemiluminescence level by calculating regression equations using the 4-parameter logistic model (weighting: 1/Y2) using the measured values of the ECL signals obtained by measuring the standard sample solutions B1 to B7 as Y and the RFD concentrations (fmol/μL) as X. Additionally, the first to third ECL signals obtained by measuring the repeatability validation sample solutions C1 to C7 were substituted into each regression equation calculated from the standard sample solutions B1 to B7, and the dystrophin assay values were calculated. The results calculated from the first to third measured values of electrochemiluminescence level are shown in Table 7 (first to third times of repeatability).

TABLE 7

| | | Sample name | | | | | | |
|---|---|---|---|---|---|---|---|---|
| | | Repeatability | Repeatability | Repeatability | Repeatability | Repeatability | Repeatability | Repeatability |
| | | validation | validation | validation | validation | validation | validation | validation |
| | | sample | sample | sample | sample | sample | sample | sample |
| | | solution C1 | solution C2 | solution C3 | solution C4 | solution C5 | solution C6 | solution C7 |
| RFD concentration | | 0.000150 | 0.000200 | 0.000300 | 0.000400 | 0.00100 | 0.00400 | 0.0100 |
| (fmol/μL) | | | | | | | | |
| Repeatability | Assay | 0.000220 | 0.000196 | 0.000396 | 0.000356 | 0.00105 | 0.00390 | 0.00991 |
| first time | value | 0.000207 | 0.000197 | 0.000376 | 0.000353 | 0.000964 | 0.00390 | 0.0101 |
| | (fmol/μL) | 0.000190 | 0.000227 | 0.000331 | 0.000374 | 0.000996 | 0.00403 | 0.00999 |
| | | 0.000163 | 0.000220 | 0.000320 | 0.000366 | 0.00104 | 0.00381 | 0.00982 |
| | | 0.000185 | 0.000264 | 0.000301 | 0.000348 | 0.00104 | 0.00396 | 0.0106 |
| | Average | 0.000193 | 0.000221 | 0.000345 | 0.000359 | 0.00102 | 0.00392 | 0.0101 |
| | value | | | | | | | |
| | (fmol/μL) | | | | | | | |
| | CV (%) | 11.3 | 12.6 | 11.5 | 2.9 | 3.6 | 2.1 | 3.0 |
| | Trueness | 128.7 | 110.4 | 114.9 | 89.9 | 101.8 | 98.0 | 100.8 |
| | (%) | | | | | | | |
| | Total | 40.0 | 23.0 | 26.5 | 13.1 | 5.4 | 4.1 | 3.9 |
| | error (%) | | | | | | | |
| Repeatability | Assay | 0.000196 | 0.000168 | 0.000339 | 0.000436 | 0.00108 | 0.00409 | 0.00919 |
| second time | value | 0.000163 | 0.000206 | 0.000352 | 0.000392 | 0.00106 | 0.00401 | 0.00983 |
| | (fmol/μL) | 0.0000819 | 0.000214 | 0.000287 | 0.000418 | 0.00119 | 0.00376 | 0.0100 |
| | | 0.0000925 | 0.000190 | 0.000329 | 0.000407 | 0.00121 | 0.00414 | 0.0102 |
| | | 0.000130 | 0.000206 | 0.000310 | 0.000430 | 0.000987 | 0.00417 | 0.0101 |
| | Average | 0.000133 | 0.000197 | 0.000323 | 0.000417 | 0.00111 | 0.00403 | 0.00986 |
| | value | | | | | | | |
| | (fmol/μL) | | | | | | | |
| | CV (%) | 36.0 | 9.3 | 7.9 | 4.3 | 8.4 | 4.1 | 4.1 |
| | Trueness | 88.5 | 98.4 | 107.8 | 104.2 | 110.5 | 100.9 | 98.6 |
| | (%) | | | | | | | |
| | Total | 47.6 | 10.9 | 15.7 | 8.4 | 19.0 | 4.9 | 5.4 |
| | error (%) | | | | | | | |
| Repeatability | Assay | 0.000146 | 0.000223 | 0.000357 | 0.000468 | 0.00100 | 0.00395 | 0.0102 |
| third time | value | 0.000160 | 0.000222 | 0.000341 | 0.000438 | 0.000984 | 0.00419 | 0.0106 |
| | (fmol/μL) | 0.000124 | 0.000207 | 0.000320 | 0.000453 | 0.00102 | 0.00410 | 0.0103 |
| | | 0.000135 | 0.000195 | 0.000349 | 0.000360 | 0.00112 | 0.00411 | 0.00991 |
| | | 0.000141 | 0.000232 | 0.000354 | 0.000438 | 0.00112 | 0.00401 | 0.0111 |
| | Average | 0.000141 | 0.000216 | 0.000344 | 0.000431 | 0.00105 | 0.00407 | 0.0104 |
| | value | | | | | | | |
| | (fmol/μL) | | | | | | | |
| | CV (%) | 9.4 | 6.8 | 4.3 | 9.7 | 6.3 | 2.3 | 4.3 |
| | Trueness | 94.1 | 107.9 | 114.7 | 107.9 | 104.9 | 101.8 | 104.2 |
| | (%) | | | | | | | |
| | Total | 15.3 | 14.7 | 19.0 | 17.5 | 11.2 | 4.1 | 8.6 |
| | error (%) | | | | | | | |
| Overall | Average | 0.000156 | 0.000211 | 0.000337 | 0.000402 | 0.00106 | 0.00401 | 0.0101 |
| (n = 15) | value | | | | | | | |
| | (fmol/μL) | | | | | | | |
| | CV (%) | 25.7 | 10.5 | 8.4 | 10.1 | 7.0 | 3.2 | 4.3 |
| | Trueness | 103.8 | 105.6 | 112.5 | 100.6 | 105.7 | 100.2 | 101.2 |
| | (%) | | | | | | | |
| | Total | 29.5 | 16.1 | 20.9 | 10.7 | 12.8 | 3.4 | 5.5 |
| | error (%) | | | | | | | |

### Intraday Repeatability Evaluation

The average value, precision (CV), trueness, and total error of the assay values of the repeatability validation sample solutions C1 to C7 were calculated for each of the first to third measurements of electrochemiluminescence level. Note that precision (%) was calculated by the following formula: (standard deviation of assay values/average of assay values)×100. Furthermore, total error (%) was calculated by the following formula: {absolute value of (trueness−100.0)}+CV.

As shown in Table 7, in the first time of intraday repeatability, precision of C1 (0.000150 fmol/μL) and C7 (0.0100 fmol/μL) was 11.3% and 3.0%, and trueness was 128.7% and 100.8%. Precision of the other standard sample solutions was from 2.1 to 12.6%, and trueness was from 89.9 to 114.9%. Considering the fact that precision of intraday repeatability in western blotting is typically from 2.9 to 33.3%, it is understood that the present measurement method is a dystrophin measurement method having excellent precision of intraday repeatability.

### Interday Repeatability Evaluation

As shown in Table 7, in interday repeatability (overall), precision of C1 (0.000150 fmol/μL) and C7 (0.0100 fmol/μL) was 25.7 and 4.3%, and trueness was 103.8 and 101.2%. Precision of the other standard sample solutions was from 3.2 to 10.5%, and trueness was from 100.2 to 112.5%. Considering the fact that precision of interday repeatability in western blotting is typically from 6.3 to 20.4%, it is understood that the present measurement method is a dystrophin measurement method having excellent precision of interday repeatability.

When the evaluation results of the above Examples 1 to 3 are comprehensively considered, the present measurement method can be said to be a dystrophin measurement method having excellent sensitivity, trueness, and precision.

### EXAMPLE 4

### Reference Example: Specificity Evaluation

### Measurement of Electrochemiluminescence Level

Electrochemiluminescence level was measured in the same manner as Example 1 except that the biological sample solution b and the biological sample solution c were used as measurement samples instead of the biological sample solutions a1 to a5. Here, the biological sample solution b (cerebellum lysate) is used as the measurement sample in Example 4, and the biological sample solution c (DMD patient muscle tissue homogenate) is used in a reference example.

### Creation of Standard Curve

Similar to Example 1, a standard curve is created by calculating a regression equation from the ECL signals obtained by measuring the standard sample solutions A1 to A9.

### Calculation of Assay Values

The ECL signals obtained by measuring the biological sample solution b and the biological sample solution c were substituted into the regression equation calculated from the standard sample solutions A1 to A9, and the dystrophin assay values were calculated. The results are shown in Table 8.

TABLE 8

| | Example 4 | Reference example |
|---|---|---|
| Sample name | Biological sample | Biological sample solution |
| | solution b | c (DMD patient muscle |
| | (Cerebellum lysate) | tissue homogenate) |
| Assay value | 0.000325 | <LLOQ |
| (fmol/μL) | | |

<LLOQ: Below lower limit of quantification

As shown in Table 8, it was possible to measure dystrophin present in the cerebellum in the case of Example 4 which used a cerebellum lysate as the biological sample.

On the other hand, in the reference example which used a DMD patient muscle tissue homogenate as the biological sample, it was below the measurement limit and dystrophin could not be measured. This is surmised to be because the dystrophin expressed in the DMD patient muscle tissue was not full-length dystrophin and lacked a C-terminal, and thus was unable to bind via the detection antibody pAb1.

In this manner, the present measurement method enables specific measurement of dystrophin.

1. A measurement method for proteins, the method comprising:
an antigen binding step of binding a protein related to a genetic disease with a capture antibody recognizing the protein;
a detection antibody binding step of binding the protein bound to the capture antibody with a detection antibody recognizing a region of the protein different from a region recognized by the capture antibody and labeled with a luminescent metal complex; and
a measurement step of detecting the detection antibody bound to the protein and labeled with the luminescent metal complex by measuring an electrochemiluminescence level generated by electrochemical stimulation.

2. The measurement method for proteins according to claim 1, wherein the capture antibody is a monoclonal antibody and the detection antibody is a polyclonal antibody.

3. The measurement method for proteins according to claim 1, wherein the capture antibody is a polyclonal antibody and the detection antibody is a monoclonal antibody.

4. The measurement method for proteins according to claim 1, wherein
the capture antibody is an antibody recognizing a C-terminal region of the protein and the detection antibody is an antibody recognizing an N-terminal region of the protein.

5. The measurement method for proteins according to claim 1, wherein
the capture antibody is an antibody recognizing an N-terminal region of the protein and the detection antibody is an antibody recognizing a C-terminal region of the protein.

6. The measurement method for proteins according to claim 1, the method further comprising:
a solubilizing step of solubilizing the protein from a biological sample using a solubilizing solution containing a surfactant and a reducing agent; and
a diluting step of diluting the solubilized sample solution obtained in the solubilizing step to obtain a dilute sample solution containing the protein; wherein,
in the antigen binding step, the protein contained in the dilute sample solution is made to bind with the capture antibody.

7. The measurement method for proteins according to claim 6, wherein, in the solubilizing step, cells or tissues derived from muscle, brain, blood, or heart, or cells or tissues cultured or derived from stem cells derived from biological cells are used as the biological sample.

8. The measurement method for proteins according to claim 6, wherein
the surfactant of the solubilizing solution used in the solubilizing step comprises an anionic surfactant.

9. The measurement method for proteins according to claim 6, wherein,
in the diluting step, the solubilized sample solution is diluted using a diluting solution comprising a nonionic surfactant.

10. The measurement method for proteins according to claim 6, the method further comprising:
a calculation step of calculating a quantity of the protein contained in the biological sample based on a standard curve of concentration and electrochemiluminescence level of a standard protein and an electrochemiluminescence level of the protein contained in the biological sample.

11. The measurement method for proteins according to claim 10, wherein a full length of the protein or a portion thereof is used as the standard protein.

12. The measurement method for proteins according to claim 1, wherein
the luminescent metal complex is introduced to the detection antibody by a detection secondary antibody recognizing the detection antibody and labeled with the luminescent metal complex.

13. The measurement method for proteins according to claim 1, the method further comprising:
a secondary antibody immobilizing step of immobilizing an immobilizing secondary antibody recognizing the capture antibody; and
a capture antibody binding step of immobilizing the capture antibody by binding the immobilized immobilizing secondary antibody with the capture antibody.

14. The measurement method for proteins according to claim 1, wherein the protein is dystrophin protein.

15. A protein measurement kit for ECL, the kit comprising:
a capture antibody recognizing a protein related to a genetic disease; and
a detection antibody recognizing a region of the protein different from the region recognized by the capture antibody and labeled with a luminescent metal complex.

16. The protein measurement kit for ECL according to claim 15, the kit further comprising:
a detection secondary antibody recognizing the detection antibody and labeled with the luminescent metal complex.
